(19) United States
(12) Patent Application Publication
(10) Pub. No.: US 2004/0039302 A1
(43) Pub. Date: Feb. 26, 2004

(54) Disposable lancing device

(75) Inventor: Yong Pil Kim, Seoul (KR)

Correspondence Address: MERCHANT & GOULD PC, P.O. BOX 2903, MINNEAPOLIS, MN, 55402-0903, US

(73) Assignee: Yong Pil Kim, Seoul (KR)

(21) Appl. No.: 10/244,055

(22) Filed: Sep. 13, 2002

(30) Foreign Application Priority Data
Aug. 23, 2002 (KR) 2002-25130 (U.M.)

(51) Int. Cl. A61B 5/00

(52) U.S. Cl. 600/583

(57) ABSTRACT

The present invention relates to a disposable lancing device which can prevent infectious possibility because the device may not be reused after a lancet is shot.

The lancing device injecting a lancet into a skin by means of the elasticity stored in a elastic member, comprises: a lancet holder having a front portion holding a lancet, a rear portion engaged with the elastic member, and a protruding portion configured to have a rectangular cross section in the bottom; a pushing member having a latch of the rectangular cross section to fix the lancet by supporting the front surface of the protruding portion of said lancet holder before using the lancet, and supporting the rear surface of the protruding portion of said lancet holder after using the lancet thereby preventing the lancet from retreating; and a body receiving the lancet holder inward and engaged with the pushing member to outward.

### BACKGROUND OF THE INVENTION

1. Field of the Invention

The present invention relates to a disposable lancing device to withdraw a blood sample for medical treatment, and more particularly, to a disposable lancing device which may not be reused after a lancet is shot, thereby preventing infectious possibility.

2. Description of the Prior Art

Generally, a lancing device is used when a small amount of blood is needed, which is used for blood type test or blood sugar test while an injector is used when a some amount of blood is needed.

FIGS. 1 and 2 are perspective and cross-sectional views of a conventional disposable lancing device. As shown in FIGS. 1 and 2, a lancet 35 is hold at the fore of a lancet holder 30. A elastic member 40 is engaged with the rear portion of the lancet holder 30. The lancet holder 30 has a protruding portion 32 at its rear portion. The protruding portion 32 is fixed by a lower end sloping side 22 which is inserted toward the inside of the body 10 and formed having a single unit with a pushing member 20 attached to the outside of a body 10. When the pushing member 20 is pushed, the sloping side 22 is tilted to release the fixed lancet holder 30. Therefore the lancet holder 30 is sprung toward due to elasticity stored in the elastic member 40. As a result, a lancet 35 hold to the fore of the lancet holder is shot from the body. However, in the conventional disposable lancing device, A user can push the lancet holder 30 back voluntarily, the sloping side 22 of the pushing member 20 can be pushed by the protruding portion 32 of the lancet holder 30. So the protruding portion 32 returns to its initial state, and the sloping side 22 of the pushing member 20 also returns to the initial state. Therefore elasticity stored in the elastic member 40, and the lancet holder 30 is fixed to the pushing member 20 the same as before using state. As a result, it is easy for the user to reuse the disposable lancing device voluntarily. In this way, if the disposable lancing device is reused, it has sanitary problems such as infection.

### SUMMARY OF THE INVENTION

In order to overcome the above-described problem, the present invention has an object to provide a disposable lancing device, which can not be reused after a lancet is shot, thereby preventing infectious possibility.

To accomplish the object, there is provided a lancing device injecting a lancet into a skin by means of the elasticity stored in a elastic member, comprising: a lancet holder having a front portion holding a lancet and a rear portion engaged with the elastic member, and a protruding portion configured to have a rectangular cross section in the bottom; a pushing member having a latch of the rectangular cross section to fix the lancet by supporting the front surface of the protruding portion of said lancet holder before using the lancet, and supporting the rear surface of the protruding portion of said lancet holder after using the lancet thereby preventing the lancet from retreating; and a body receiving the lancet holder inward and engaged with the pushing member to outward.

There is also provided the said lancing device, wherein the body has a cap at the fore for guiding the lancet forward and adjusting the gap between the body and said cap by rotation.

### BRIEF DESCRIPTION OF THE DRAWINGS

The present invention will be described in terms of preferable embodiments with reference to the accompanying drawings, which are given only by way of illustration and thus are not limitative of the present invention, wherein:

FIG. 1 is a perspective view of a conventional disposable lancing device;

FIG. 2 is a cross-sectional view of the conventional disposable lancing device;

FIG. 3 is a exploded perspective view of a disposable lancing device in accordance with the present invention;

FIG. 4a is a perspective view illustrating the state before use of the disposable lancing device in accordance with the present invention;

FIG. 4b is a perspective view illustrating the state after use of the disposable lancing device in accordance with the present invention;

FIG. 5a is a cross-sectional view cut according to A-A line of FIG. 4a; and

FIG. 5b is a cross-sectional view cut according to B-B line of FIG. 4b.

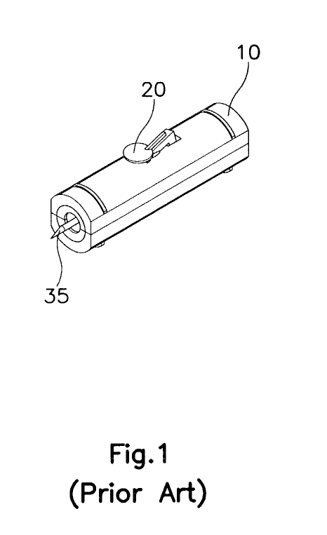

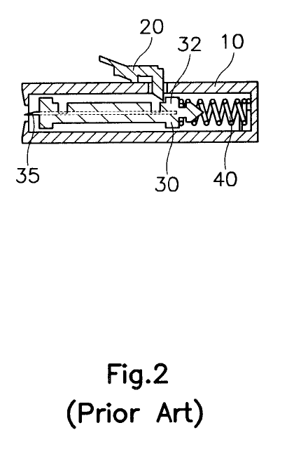

### DETAILED DESCRIPTION OF THE PREFERRED EMBODIMENTS

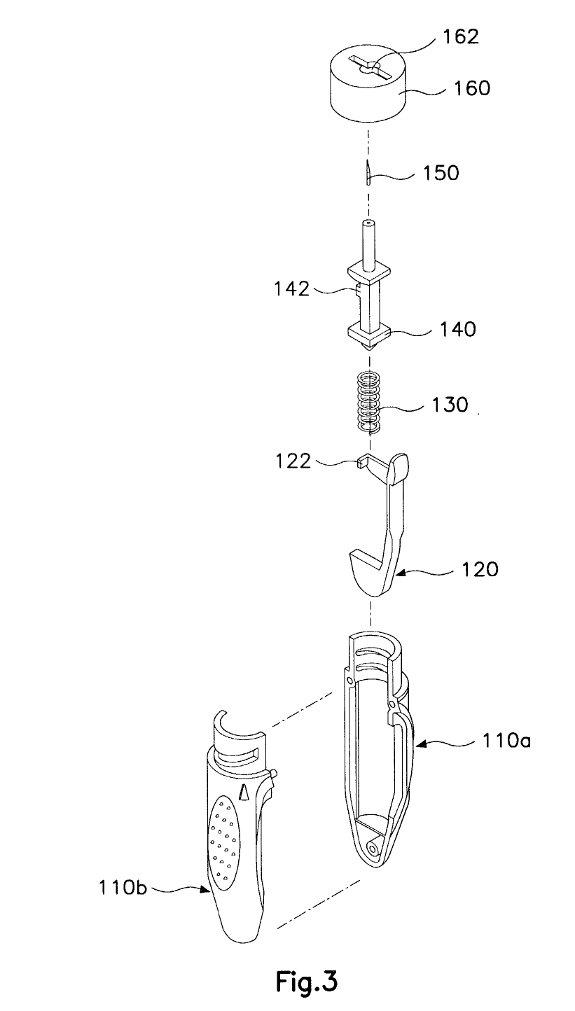

FIG. 3 is a exploded perspective view of a disposable lancing device in accordance with the present invention.

As shown in FIG. 3, a disposable lancing device according to the present invention comprises a lancet holder 140, a pushing member 120, a body 110a and 110b and a cap 160. The lancet holder 140 has a lancet 150 attached to the fore and a rear portion engaged with a elastic member 130. The pushing member 120 regulates the operation of the lancet holder 140. The body 110a and 10b are receiving the lancet holder 140 and the pushing member 120. The body 110a and 110b has the cap 160 at the fore (at the upper side of FIG. 3 for guiding the lancet forward and adjusting the gap between the body by rotation.

The lancet holder 140 is configured to be able to move forward and backward within the body 110a and 110b. One end of the elastic member 130 engaged with the rear portion of the lancet holder 140 and the other end of said elastic member is fixed with the body. If the lancet holder 140 is pushed backward within the body 110, the elastic member 130 stores elasticity. In order to maintain the state, a protruding portion 142 formed at a certain portion of the lancet holder 140 is fixed by latch 122 of the pushing member 120.

The pushing member 120 has the latch 122 drawn to the inside of the body 110a and 110b and bent toward the bottom side of the lancet holder 140. The latch 122 is configured to have a rectangular cross section, and the protruding portion 142 fixed by the latch 122 is configured to have a rectangular cross section. As a result, both the latch 122 and the protruding portion 143 have rectangular cross sections respectively, the latch 122 of the pushing member 120 can be supported by the front and rear sides of the protruding portion 142. Therefore the lancet holder 140 having the lancet 150 cannot be pushed back, thereby preventing reuse of the lancet.

The body is made of two members 110a and 10b. An opening 112 is formed at the front of the body, and the pushing member 120 is engaged with the rear portion of the body. A helical hole 114 is formed one side of the opening 112, where the projected portions of the cap 160 is inserted. So rotation of the cap 160 causes change the gap between the body 110 and the cap 160, that is, the length of the lancet 150 to be shot from the cap 160. As a result, an injected depth of the lancet 150 into a skin can be adjusted.

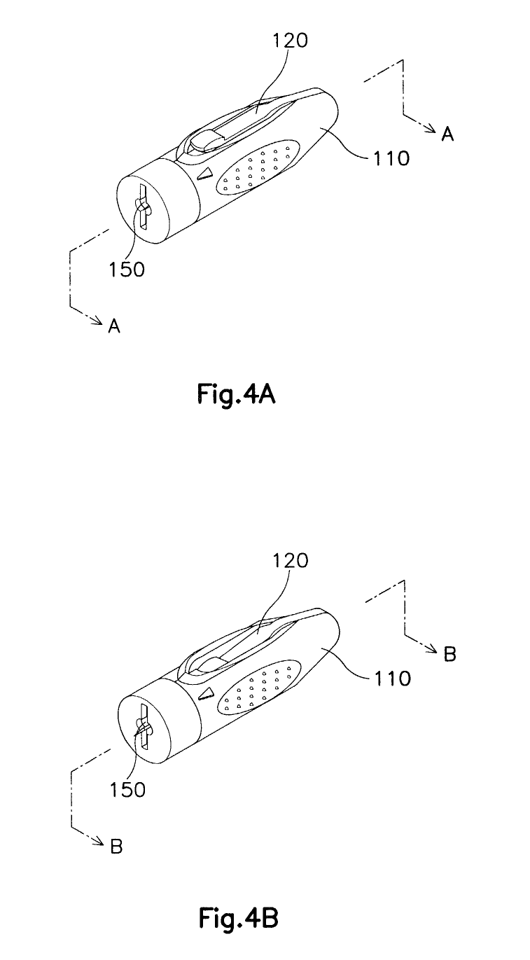

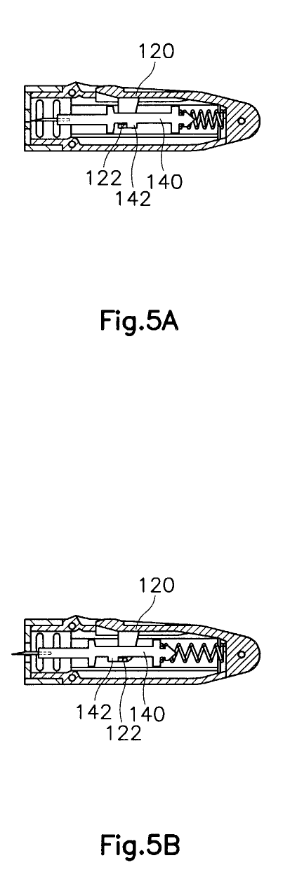

FIGS. 4a and 5a are perspective and cross-sectional views illustrating the state before use of the disposable lancing device in accordance with the present invention.

As shown in FIGS. 4a and 5a, the disposable lancing device of the present invention comprises the body 110 receiving the lancet 150, the pushing member 120 formed on one portion of the body, and the cap 160 having a hole 162 through which the lancet 150 can be shot.

Before use of the lancing device, the elastic member engaged with the rear portion of the lancet holder 40 is contracted with elasticity, while the latch 122 of the pushing member 120 supports the front side of the protruding portion 142 of the lancet holder 140. If the pushing member 120 is pushed, the latch 122 moves down in parallel and no longer supports the protruding portion 142 of the lancet holder 140. So, the lancet is shot toward (leftward of Figs.) due to the elasticity stored in the elastic member. A user places the hole 162 of the cap 160 on the skin to be shot, and pushes the pushing member 120,then the lancet 150 is shot through the hole 162 of the cap 160 due to the elasticity of the elastic member and pierces the skin.

FIGS. 4b and 5b are perspective and cross-sectional views illustrating the state after use of the disposable lancing device in accordance with the present invention.

After use of the lancing device, as shown FIG. 4b, the lancet is ejected from the hole and then fixed. The conventional lancing devices have the problem that the ejected lancet 150 can be loaded by the tilting of the said sloping side. On the other hand, in the lancing device of the present invention, the protruding portion 142 of the lancet holder 140 and latch 122 of the pushing member 120 have both rectangular cross section. After the lancet 150 is shot, the latch 122 of the pushing member supports the rear portion of the protruding portion 142 of the lancet holder 140. As a result, the lancet holder 140 cannot be retreated. Accordingly, the lancet cannot be reused.

As discussed earlier, the disposable lancing device of the present invention can prevent the reuse of the lancet because it is formed not to be retreated the lancet into the body after the lancet is shot.

What is claimed is:

1. A lancing device injecting a lancet into a skin by means of the elasticity stored in a elastic member, comprising:
a lancet holder having a front portion holding a lancet, a rear portion engaged with the elastic member, and a protruding portion configured to have a rectangular cross section in the bottom;
a pushing member having a latch of the rectangular cross section to fix the lancet by supporting the front surface of the protruding portion of said lancet holder before using the lancet, and supporting the rear surface of the protruding portion of said lancet holder after using the lancet thereby preventing the lancet from retreating; and
a body receiving the lancet holder inward and engaged with the pushing member to outward.

2. The lancing device according to claim 1, wherein the body has a cap at the fore for guiding the lancet forward and adjusting the gap between the body and said cap by rotation.
